SEQUENCE LISTING (4 sequences)

SEQ ID NO: 1 - DNA, 25 bp, Artificial Sequence
  Primer EB08
gcgcttcgcgcagatcagaaatccc

SEQ ID NO: 2 - DNA, 24 bp, Artificial Sequence
  Primer EB11
atcaaggaatggggcatcacctag

SEQ ID NO: 3 - RNA, 19 nt, Artificial Sequence
  Inverted repeat to the luciferase gene of Photinus pyralis
cuuacgcugaguacuucga

SEQ ID NO: 4 - RNA, 19 nt, Artificial Sequence
  Inverted repeat to the luciferase gene of Photinus pyralis
ucgaaguacucagcguaag

(19) United States
(12) Patent Application Publication
(10) Pub. No.: US 2005/0260166 A1
(43) Pub. Date: Nov. 24, 2005

(54) Expressional enhancers from viruses

(75) Inventors: Marinus Willem Prins, Randwijk (NL); Robert Willem Goldbach, Wageningen (NL); Petrus Theodorus de Haan, Oegstgeest (NL); Gerrit Jan van Holst, Leiden (NL)

Correspondence Address: TRASK BRITT, P.O. BOX 2550, SALT LAKE CITY, UT, 84110, US

(73) Assignee: Phytovation B.V., Enkhuizen (NL)

(21) Appl. No.: 11/107,315

(22) Filed: Apr. 15, 2005

Related U.S. Application Data
(63) Continuation of application No. PCT/NL03/00694, filed on Oct. 15, 2003.

(30) Foreign Application Priority Data
Oct. 15, 2002 (EP) 02079257.8

(51) Int. Cl. A61K 48/00; C12N 15/86

(52) U.S. Cl. 424/93.2; 435/456

(57) ABSTRACT

The present invention discloses that vertebrate viruses encode RNA silencing suppressors/expressional enhancers which enable them to overcome host intracellular defense responses. This has paved the way for the development of protein production systems and for production systems of (recombinant) virus particles, and of vaccines directed against vertebrate viruses.

### CROSS-REFERENCE TO RELATED APPLICATIONS

This application is a continuation of PCT International Patent Application No. PCT/NL2003/000694, filed on Oct. 15, 2003, designating the United States of America, and published, in English, as PCT International Publication No. WO 2004/035796 A1 on Apr. 29, 2004, which application claims priority to European Patent Application No. 02079257.8, filed Oct. 15, 2002, the contents of the entirety of each of which are incorporated herein by this reference.

### TECHNICAL FIELD

The invention relates to the field of molecular biology, protein production (pharmaceuticals, industrial enzymes, etc.), (recombinant) virus production, and vaccine development. More specifically, it relates to the identification of a protein or fragment thereof or RNA molecule of a vertebrate virus which can act as an RNA silencing suppressor and, therefore, also as an expressional enhancer. In particular, the invention paves the way for a) enhanced transgene expression, b) the production of vaccines directed against viruses, c) the production of live vaccines directed against viruses, and, d) the production of recombinant viral vector particles.

### BACKGROUND

Viruses, as intracellular parasites, make use of the host cell machinery for their replication. To do this, they have genomes and expression strategies that mimic that of the host. Viral genomes encode viral structural proteins and non-structural proteins. The structural proteins are included in the virus particles and comprise coat proteins, nucleocapsid or core proteins, matrix proteins and/or membrane glycoproteins. Non-structural proteins include proteins involved in RNA transcription and replication, RNA-dependent RNA polymerases and helicases. Another class of viral non-structural proteins is those that regulate gene expression in infected cells. (Fields B. N., et al. (eds.), Virology, Lippincott Williams & Wilkins Publishers, 2001).

At least seven supergroups of eukaryotic RNA viruses are presently recognized. The most homogeneous supergroup is formed by the negative-strand RNA viruses, which include the family Rhabdoviridae, Filoviridae, Paramyxoviridae, Bornaviridae, Orthomyxoviridae, Bunyaviridae, Arenaviridae and the genus Tenuivirus.

The majority of negative-strand RNA viruses has an animal host. Members of only two genera, Tospovirus and Tenuivirus have plant hosts.

The tospoviruses form a genus of phytopathogenic viruses within the arthropod-borne family of Bunyaviridae. The type species of the genus Tospovirus, Tomato spotted wilt virus (TSWV), has a very broad host range, encompassing more than 800 plant species belonging to 82 different families, including many important crops and ornamentals. Tenuiviruses predominantly infect monocot plant species. They share some similarities with viruses classified in the Family Bunyaviridae, particularly with those in the genus Phlebovirus. The number of RNA segments and the apparent lack of enveloped virus particles distinguishes Tenuiviruses from viruses in the Family Bunyaviridae.

Animal positive strand RNA viruses are much more diverse than the negative-strand RNA viruses. Seven families have members infecting mammals including man: Coronaviridae, Arteriviridae, Togaviridae, Flaviviridae, Picornaviridae, Caliciviridae and Astroviridae. The order of Nidovirales shares a number of properties with the non-segmented negative-strand RNA viruses. The Nidovirales comprises two virus families, the Arteriviridae and Coronaviridae, which is subdivided into the genus Coronavirus and the genus Torovirus.

The Togaviridae are divided into two genera: Alphavirus (with 18 members including Sindbis, Semliki Forest and Ross River virus) and Rubivirus (with 1 member, Rubella virus). The family Flaviviridae is divided into three genera, Flavivirus, Pestivirus and Hepacivirus.

The family Picornaviridae is divided into six different genera: Aphtovirus, Parechovirus, Cardiovirus, Enterovirus, Hepatovirus, and Rhinovirus, from which three of them, Enterovirus, Rhinovirus and Hepatovirus, include relevant human pathogens. The Retroviridae are sexually transmitted viruses and include the human T-cell Lymphotrophic virus type I (HTLV-I), causing myelopathy and tropical spastic Paraparesis and human immune deficiency virus (HIV), the causal agent of the acquired immune deficiency syndrome (AIDS).

The Reoviridae are double strand RNA viruses. Reovirus infections in humans are typically asymptomatic, but ubiquitous.

Vertebrate viruses with DNA genomes are highly divergent. The Hepadnaviridae comprise two genera, from which Hepatitis B virus (HBV) within the genus Orthohepadnavirus causes hepatitis in humans.

The Papovaviridae comprise Papillomaviruses and Polyomaviruses. The human papilloma virus is involved in the formation of cervix carcinomas. Polyomaviruses are mostly asymptomatic.

Adenoviruses comprise many different serotypes, which cause upper respiratory, intestinal, and eye infections in humans.

The Poxviridae comprise a large family of enveloped viruses with many members infecting a wide range of animals. The human variola virus causing smallpox has been eradicated using Vaccinia virus as a vaccine strain.

Herpes viruses from all species have been classified into three groups based upon tissue tropism, pathogenicity, and behavior. Alpha herpes viruses are usually fast replicating and, in humans, are represented by Herpes Simplex virus-1 and 2, and Varicella Zoster Virus. The slowly replicating beta herpes viruses are represented by Cytomegalovirus and Human Herpes virus-6 and 7. Gamma herpes viruses are poorly replicating and readily transform cells. The gamma herpes viruses in humans are represented by the Epstein-Barr Virus (EBV), which is a B-cell transforming virus and the causative agent of infectious mononucleosis, and the Human Herpes virus-8 (HHV-8) or Kaposi's sarcoma associated virus (KSHV) present in Kaposi's sarcoma.

Eukaryotic hosts have several lines of defense against virus infections, acting at the level of viral entry, replication or spread. A first line of defense consists of physical/chemical barriers, blocking viral entry. A second line of defense comprises the innate responses, inhibiting virus replication. The innate responses include the interferon response in animals and the systemic acquired resistance (SAR) response in plants. It has been demonstrated in plants that RNA silencing should be considered as an innate defense response. A third line of defense in animals comprises immune responses mediated by natural killer cells, T-lymphocytes, and antibodies and are directed against virus replication and spread often by inducing cell death of infected cells. Plants lack specialized immune cells and here the third line of defense comprises disease resistance (R) proteins which activate hypersensitive responses upon pathogen recognition including localized cell death.

Gene expression in eukaryotic cells is controlled by several regulatory mechanisms acting at the transcriptional level in the nucleus or at the post-transcriptional level in the cytoplasm. Recently, a novel regulatory mechanism has been identified referred to as RNA silencing in which RNAs instead of proteins serve as signaling and target molecules. RNA silencing has been proven to be induced by over-expressed and double-stranded RNA molecules and results in RNA degradation in the cytoplasm of cells from higher eukaryotes, thereby silencing expression of proteins (Ding, 2000, Curr. Opin. Biotechnol. 11: 152-156). It is thought that RNA silencing has been developed by eukaryotes to prevent expression of alien/foreign genetic information such as transposable elements and viruses. In this respect, RNA silencing represents an innate intracellular defense mechanism. Over recent years, silencing has been described in an increasing variety of organisms and referred to as quelling in fungi or RNAi in animals. Recently, RNA silencing has also been observed in insects. Three main methods have been employed to silence gene expression in eukaryotes, namely anti-sense silencing, sense co-suppression and RNA interference (RNAi), otherwise known as post transcriptional gene silencing (PTGS).

In anti-sense silencing, it was hypothesized that the expression product (antisense mRNA) interferes with the expression product of a homologous endogenous gene (sense mRNA) thereby, inhibiting translation. In sense co-suppression, it has been found that the expressed product (sense mRNA), sometimes does not lead to (over) expression of the product for which it codes but in some way interferes with the endogenous (homologous) gene (or its mRNA). RNAi or PTGS refers to inhibition of gene expression by the presence of homologous double-stranded RNA (dsRNA) structures. It has now been generally accepted that a single general mechanism referred to as RNA silencing underlies all these observed silencing phenomena. Double-stranded (ds) RNA is either introduced into a cell or, in the case of anti-sense expression and sense-cosuppression, over-expressed RNA is made double-stranded by a host-encoded RNA-dependent RNA polymerase (RdRp). The central initiator molecules in RNA silencing are dsRNA molecules, which are degraded by a dsRNA-targeted nuclease, denoted DICER to 21-23 nt fragments. These short interfering RNAs (siRNAs) subsequently target homologous cognate RNAs for degradation mediated by the RNA-initiated silencing complex (RISC), resulting in a low amount of cytoplasmic target RNA, while transcription in the nucleus is maintained. Next to the RdRp and DICER, additional host genes have been found in coding for proteins involved in RNA silencing, such as an eIF2C transcription factor-like protein (AGO1), an RNA helicase (GEMIN3, SDE3) and an accessory factor (SGS3). Although required for successful silencing, their precise role in RNA silencing has not yet been elucidated.

The siRNA species also induce RNA-directed DNA methylation of homologous sequences in the nucleus. DNA methylation leads to a closed chromatin conformation, wherein transcription of the affected nuclear DNA is suppressed. Hence, the siRNAs and the RNA silencing machinery play also a role in a process referred to as transcriptional gene silencing (TGS), which is characterized by decreased transcription of the homologous gene in the nucleus.

Explanted animal somatic cells grown as monolayers in tissue culture possess an intrinsically programmed limit to their capacity for proliferation, referred to as the Hayflick limit. Cell cultures derived from cancer tissues once established in tissue culture are often immortal, which means that they can be cultured indefinitely. It is generally thought that immortalization represents a single step in the multi-step nature of the cell transformation process. In 1994, Kim et al., Science 266: 2011-2016 demonstrated that human cancer cells possess telomerase activity, which is absent in normal mortal somatic tissues. Telomerase is involved in maintaining telomeres at the ends of chromosomes, through the synthesis of characteristic telomere repeat sequences. In primary cell lines, which lack telomerase activity, the telomeres progressively shorten with each division cycle, leading to the replicative senescence that characterizes the Hayflick limit. Transfection of an expression vector encoding the human telomerase into human fibroblasts leads to immortalization of these cells. These immortal cells have elongated telomeres, normal karyotypes and do not express markers of malignancy (Jiang et al., Nature Genetics 21:111-114, 1999; Morales et al., Nature Genetics 21:115-118, 1999). The RNA silencing machinery is involved in chromosome dynamics during mitosis and meiosis and is responsible for suppression of the telomerase expression in mortal cells (Newbold, Mutagenesis 17: 539-550, 2002).

It has become clear from studies in nematodes and plants, that RNA silencing in eukaryotes regulates differentiation processes. RNA silencing inhibits replication of transposable elements and viruses. In this respect, RNA silencing can be observed as an intracellular innate defense mechanism. As part of the ongoing battle between parasites and hosts, viruses evolve mechanisms to overcome the innate and /or cellular responses. To counteract the Interferon response, a number of animal viruses encode interferon antagonists (IA). Patent application WO 01/77394 describes a method to identify viral IA proteins and their use in isolating various types of attenuated viruses, having an impaired ability to antagonize the interferon response. In addition, a method is described how to use such IA proteins for the identification of new antiviral agents.

The majority of economically relevant viruses and attenuated strains thereof grow well in cells lacking the interferon response, such as Vero cells. However, even in cells lacking an interferon response, virus replication and especially that of attenuated strains is still inhibited by the other innate response, e.g., RNA silencing.

To overcome host RNA silencing, it has been found that a number of plant viruses encode RNA silencing suppressors (RSS). PCT International patent publications WO 98/44097, WO 01/38512, and WO 02/057467 deal with plant virus-derived RNA silencing suppressors and their use in plants.

WO 02/057301 deals with a plant virus-derived RSS and its use in animal cells, whereas: Li et al., Science 296: 1319-1321, 2002, describes an insect virus-derived RSS and its activity in plant and insect cells.

A need exists to circumvent host-induced RNA silencing in eukaryotes, to enhance gene expression for the production of proteins including pharmaceutical proteins, monoclonal antibodies and enzymes and metabolites synthesized by proteins or via enzymes or to improve the production of viruses for the development of more cost-effective vaccines, or of recombinant viruses for use in vaccines or in gene therapies.

### SUMMARY OF THE INVENTION

The current inventors used a plant-based assay and developed mammalian cell-based assays to identify virus-derived RSS proteins or RNA molecules. Surprisingly, using these assays a number of RSS proteins or RNA molecules of vertebrate viruses have been identified, which act as suppressors of RNA silencing. RSS proteins or RNA molecules play an important role in virus replication, since they enable the virus to overcome the innate RNA silencing response in their hosts.

Remarkably, upon expression in mammalian cells with or without interferon response, these RSS proteins or RNA molecules enable viruses to grow at higher titers. As the RNA silencing machinery is involved in chromosome dynamics during mitosis and meiosis and is responsible for suppression of the telomerase expression in mortal cells (Newbold, Mutagenesis 17: 539-550, 2002), RSS proteins or RNA molecules have the capacity to immortalize primary cells by enhancing the telomerase expression. This opens the way to produce immortal cell lines from primary cells, suitable for the production of viruses, mutant or recombinant strains thereof or of viral vectors, by expressing an RSS protein or RNA molecule in the cultured primary cell.

This invention also provides a method to prevent silencing of expression of a nucleic acid in an eukaryotic cell and/or to reverse silencing of expression of a nucleic acid in an eukaryotic cell, once established and/or to enhance/boost the expression of a nucleic acid, or accumulation of its product, in an eukaryotic cell, comprising introducing into the eukaryotic cell a protein or fragment thereof or RNA molecule of a virus, which is capable of interfering with RNA silencing in the eukaryotic cell.

The current invention also provides a method to use a protein or fragment thereof or RNA molecule of a vertebrate virus, which is capable of interfering with RNA silencing in an eukaryotic cell to enhance/boost and/or stabilize the expression of a nucleotide sequence encoding a (pharmaceutical) protein, a (therapeutic) monoclonal antibody, a virus or viral vector or an (industrial) enzyme and the like.

A vertebrate virus comprises a virus from the family Arenaviridae, Bunyaviridae, Orthomyxoviridae, Bornaviridae, Filoviridae, Paramyxoviridae, Rhabdoviridae, or the genus Tenuivirus, the family Picomaviridae, Flaviviridae, Togaviridae, Coronaviridae, Arteriviridae, Caliciviridae, Astroviridae, Herpesviridae, Reoviridae, Adenoviridae, Papovaviridae or Poxviridae. In particular, the protein or fragment thereof of the invention comprises a viral non-structural protein. Preferably, the protein comprises the non-structural protein (NSs) of the genus Tospovirus within the Bunyaviridae, the non-structural protein (NS3) of the Tenuiviruses, the non-structural protein (NS1) of the Orthomyxoviridae, preferably that of influenza virus A, a non-structural protein VP35 of the Filoviridae, or a non-structural protein E3L of the Poxviridae.

In a preferred embodiment, the invention provides use of a protein or fragment thereof or RNA molecule of a plant or animal virus which is capable of interfering with RNA silencing in a fungal cell to enhance/boost and/or stabilize the expression of a nucleotide sequence encoding a pharmaceutical or other protein, a therapeutic or other monoclonal antibody, a virus or viral vector or an industrial or other enzyme, and the like or metabolites synthesized by proteins or via enzymes.

The invention further provides a method to use a protein or fragment thereof or RNA molecule of a plant or animal virus which is capable of interfering with RNA silencing in an animal host cell for the production of a virus or a mutant or recombinant strain thereof or a viral vector. More specifically, the animal host cell is derived from an existing animal cell line or from immortalized primary animal cells. Preferably, a protein or fragment thereof or RNA molecule of a plant or animal virus which is capable of interfering with RNA silencing in an animal host cell is used to immortalize primary animal cells. In another aspect, the invention provides a method of producing attenuated viruses comprising deletion or making inoperable in any other way the gene coding for the silencing suppressor in a virus. According to the invention, production of such attenuated viruses comprises generating the attenuated virus particles in a host in the presence of a protein or fragment thereof or RNA molecule of a virus.

Further, the protein or RNA molecule of the invention can be used for the improvement of packaging/producer cell lines for the production of viral vectors, including retro-, lenti-, baculo-, adeno-, adeno-associated (AAV) or hybrid virus particles such as adeno-AAV viruses. This invention preferably deals with the production of recombinant virus particles, which harbor a nucleotide construct which is able to produce a single transcript or multiple transcripts capable of folding into double-stranded RNA.

### LEGENDS TO THE FIGURES

FIG. 1. Agrobacterium infiltration experiments with different TSWV genes in N. benthamiana plants. Agrobacterium strains harboring TSWV genes are co-infiltrated with pBIN-GFP. Only TSWV NSs suppresses the silencing of GFP (panel D).

FIG. 2. RNA silencing suppression activity displayed by TSWV NSs, RHBV NS3 and Influenza virus A NS1. The photograph is taken with a yellow filter 6 days after infiltration.

FIG. 3. The effect of Influenza virus A NS1 and mutant NS1rb proteins on RNA silencing of GFP in Nicotiana benthamiana leaves. (A) UV photography of Agrobacterium infiltrated leaves. From left to right non-infiltrated wild type and subsequently pBIN-GFP is respectively co-infiltrated with an empty pBIN19 vector; pBIN-IVA-NS1; pBIN-IVA-NS1rb; pBIN-CABMV-HC-Pro and pBIN-TSWV-NSs. (B) Quantitative Western blot analysis is performed on total protein extracted from infiltrated leaf sectors, using anti-GFP antibodies. Rubisco protein abundance in these green leaves is used as a loading control and is visualized using anti-rubisco antibodies. (C) Northern blot analyses of mRNA purified from infiltrated leaf sectors, the blots are probed with a DIG-labeled GFP-specific PCR fragment. Ethidium bromide staining of the same gel shows the 25S rRNA as a loading control. (D) GFP siRNAs are extracted from infiltrated leaf sectors and detected using a DIG labeled GFP specific probe.

FIG. 4. Gel retardation studies of siRNAs binding to increasing amounts of NS1 protein. (A) Radiolabeled synthetic siRNAs (2 pM) incubated with 0, 25, 50, 100 and 200 pM of purified NS1 (lanes 1-5) and NS1rb (lanes 6-10) visualized by radiography after native gel electrophoresis. (B) Radiolabeled purified plant siRNAs incubated under the same conditions.

### DETAILED DESCRIPTION OF THE INVENTION

### DEFINITIONS

RNA silencing: A eukaryotic gene regulation mechanism in which RNA molecules instead of proteins serve as signaling and target molecules. RNA silencing is induced by over-expressed and double-stranded RNA molecules and involves sequence-specific RNA degradation (post transcriptional gene silencing, PTGS) and chromatin remodeling in the nucleus (transcriptional gene silencing, TGS).

Vertebrate virus: A virus that infects members of the subphylum Vertebrata (e.g., Mammalia, Aves, Reptilia, Amphibia, Pisces, Marsipobranchia and Leptocardia). The definition of a vertebrate virus as used in this specification includes all negative-strand RNA viruses (including plant negative-strand viruses) and all positive strand RNA viruses, double strand RNA viruses and DNA viruses which can infect members of the subphylum Vertebrata.

Heterologous nucleotide sequence: Any nucleic acid that is positioned in a place where it would not normally occur in nature. Thus, heterologous DNA can be DNA of a species foreign to the host species in which it is located, or it can be of the same species, but differently regulated or else dislocated. It can be DNA/RNA from a different virus or from the same virus but differently regulated or dislocated.

Heterologous protein: A protein derived from a different species.

Homologous protein: A protein derived from the same species.

Transgene: A heterologous nucleic sequence integrated into the chromosomal DNA of cells from an organism.

Nucleic acid: As used herein, refers to an oligonucleotide or polynucleotide, oligoribonucleotide, polyribonucleotide, and fragments or portions thereof, and to DNA or RNA of genomic or synthetic origin which may be single- or double-stranded, and which may be of sense or antisense polarity.

RNA aptamer: A polyribonucleotide with a specific function selected from a library of oligo- or polyribonucleotide molecules.

Protein: A peptide or amino acid sequence (i.e., combinations of amino acids in peptide linkages).

Non-structural protein: A viral protein, which is not part of the viral envelop and/or capsid or nucleocapsid and usually not included in virus particles.

Ambisense RNA: An RNA molecule with a coding domain in messenger-sense (+) and anti-messenger-sense (−).

Deletion: A change in either nucleotide or amino acid sequence in which one or more nucleotides or amino acid residues, respectively, are absent.

Transformation: A process by which the genetic material carried by an individual cell of a host is altered by incorporation of a heterologous nucleotide sequence into its genome. For example, using Agro infiltration (the infiltration of Agrobacterium tumefaciens into a host) and like approaches or physical methods (gene gun, electroporation, inoculation, injection) for introducing nucleic acids into cells.

Transfection: A process by which exogenous nucleic acid in solution is introduced into eukaryotic cells. For example, using chemical (cationic lipid or polymer) and physical methods (gene gun, electroporation, inoculation, injection) and like approaches for introducing nucleic acid into cells.

Production: The large-scale manufacture of biomolecules such as proteins, metabolites and virus particles.

Attenuated virus: A weakened virus that is no longer or less virulent.

RNA silencing suppressor or A protein or fragment thereof or RNA molecule of a virus, which is capable of interfering with RNA silencing in an eukaryotic cell: A protein or an RNA molecule which in an assay, according to the invention, is found to be increasing the expression of the reporter protein, thereby indicating its capability to act as an RNA silencing suppressor. Examples of such proteins are the non-structural protein (NSs) from TSWV of the genus Tospovirus within the Bunyaviridae, the non-structural protein (NS3) from Rice hoja blanca virus (RHBV) of the genus Tenuivirus and the non-structural protein (NS1) from Influenza virus A of the Orthomyxoviridae, the non-structural protein VP35 from Ebola virus (EV) of the Filoviridae, and the non-structural protein E3L from Vaccinia virus (VV) of the Poxviridae. The definition of a RNA silencing suppressor as used in this specification includes also all proteins or RNA molecules of other than viral origin that act as an RNA silencing suppressor.

In this invention, an assay is used for identifying a protein or fragment thereof or RNA molecule of a virus, which is capable of interfering with RNA silencing in an eukaryotic cell, comprising introducing simultaneously or separately in an eukaryotic cell:
(a) a nucleic acid construct comprising a nucleotide sequence encoding a protein or RNA molecule of a virus; and
(b) a nucleic acid construct comprising a nucleotide sequence encoding a reporter protein into an eukaryotic cell silenced in the expression of a nucleic acid encoding the reporter protein, and
detecting the suppression/dampening/reversal of silencing and/or a boost in reporter protein expression. Reoccurrence of the expression of the reporter protein and a further boost of the reporter gene expression compared with controls allows one to determine if the protein has an RNA silencing suppressor and hence expressional enhancer function.

A reporter nucleic acid, as used herein, can be, for example, a nucleic acid expressing the green fluorescent protein (GFP) from jellyfish, or any other reporter such as other fluorescent proteins, luciferase from Photinus pyralis or Renilla reniformis or GUS. The GFP nucleic acid, when expressed, produces a protein that produces a green glow under ultraviolet light.

The examples show that it is possible to identify silencing suppressing proteins or RNA, because when in plants a nucleotide sequence encoding a protein of a virus was expressed together with a nucleotide sequence encoding a reporter (GFP), it showed that in some cases silencing of GFP transgene expression in plants was at least, in part, suppressed, indicating that the protein is a suppressor of RNA silencing. Furthermore, in the same or in other cases, GFP expression was shown to be boosted indicating that the viral protein acts as an RNA silencing suppressor and, hence, is also an expressional enhancer.

In another aspect, the assay comprises introducing simultaneously or separately a nucleic acid construct comprising a nucleotide sequence encoding a protein or RNA molecule of a virus and a nucleic acid construct comprising a nucleotide sequence encoding a reporter protein into a eukaryotic cell and detecting a boost in reporter protein expression.

Preferably, the assay comprises a combination of a nucleotide sequence encoding the luciferase reporter protein and a nucleotide sequence encoding a protein or RNA molecule of a virus in mammalian cells, such as HeLa, CHO, HEK293, Vero and the like, using transfection.

In another aspect, the assay comprises introducing simultaneously or separately a nucleic acid construct comprising a nucleotide sequence encoding a protein or RNA molecule of a virus and a nucleic acid construct comprising a nucleotide sequence encoding a reporter protein and a nucleic acid or nucleic acid comprising a nucleotide construct encoding a nucleic acid, which silences the expression of the reporter protein, into an eukaryotic cell, and detecting the reversal of silencing of the reporter protein.

Preferably, a kit for such an assay comprises a combination of a nucleotide sequence encoding the luciferase reporter protein (or any other reporter protein) and short interfering RNA, siRNA, molecules directed against the luciferase sequence (or sequence encoding another reporter protein) or a nucleotide sequence encoding a short hairpin RNA directed against the luciferase sequence (or sequence encoding another reporter protein) and a nucleotide sequence encoding a protein or RNA molecule of a virus capable of suppression of silencing in mammalian cells. More specifically, such mammalian cells are HeLa, CHO, HEK293 or Vero cells.

Using the assays, it is herein shown that a vertebrate virus encodes a protein or RNA molecule which can act as a suppressor of RNA silencing in eukaryotes. The protein or RNA molecule is herein shown to inhibit RNA silencing in a eukaryotic cell. Without being bound to theory, it is postulated that a protein or fragment thereof or RNA molecule of a vertebrate virus of the present invention can interact with a component of the host RNA silencing apparatus or with a cellular factor that on its turn regulates a component of the RNA silencing machinery, and as a result, RNA silencing of the eukaryotic cell is, at least in part, inactivated. This, in turn, protects RNA molecules from being degraded and as a consequence, the expression of a nucleotide sequence in the eukaryotic cell is enhanced and/or boosted.

It is also herein shown that a protein or fragment thereof or RNA molecule of a vertebrate virus can reverse existing nucleic acid-induced silencing in an eukaryotic cell, that is the expression of the nucleic acid, which has been “switched off” through RNA silencing is, at least in part, “switched on” (i.e., restored). Thus, a protein or RNA of the present invention can “block” or interfere with RNA silencing in the eukaryotic cell instigated as a result of expression of the nucleic acid.

It is to be understood that a fragment of the protein of the invention is a fragment that can be obtained by deletion of a part of the protein, wherein the fragment is still able to achieve the same function as the total protein, which function especially is the function as disclosed in this application, which is the capability to prevent or reverse RNA silencing in a host.

Numerous examples now exist in eukaryotic organisms where, upon insertion of a transgene containing a sequence homologous to an endogenous gene, the expression of both the transgene and endogenous gene is impaired and/or silenced.

The Examples show that when a protein of a vertebrate virus of the present invention is expressed in eukaryotic cells, it enhances expression of a nucleic acid construct comprising a nucleotide sequence encoding a reporter protein by interfering with RNA silencing. In addition, in human cells harboring a single non-silenced transgene copy encoding luciferase, in which the luciferase coding sequence is expressed under transcriptional control of the human EF1-alfa promoter, the luciferase expression in the cells is not significantly increased by transiently co-expressing a protein of a vertebrate virus of the present invention. Remarkably, an increase in luciferase expression is only obtained in transgenic cells in which the luciferase gene is (partly) silenced, e.g., in cells in which the luciferase coding sequence is expressed from multiple transgene copies, under transcriptional control of a strong viral promoter, such as the CMV immediate early promoter.

It is shown in the Examples that when a nucleic acid construct comprising a nucleotide sequence encoding a protein or RNA of a vertebrate virus of the present invention is simultaneously introduced into a eukaryotic cell along with a nucleic acid construct comprising a nucleotide sequence encoding a reporter protein, there is a boost in expression of the reporter protein. Thus, a viral RNA silencing suppressor/expressional enhancer of the present invention can be used for enhancing transgene expression, which can comprise the production of proteins of interest (e.g., pharmaceutical proteins, monoclonal antibodies, industrial enzymes and the like) in eukaryotic cells (e.g., animal (including vertebrate and insect), fungal (inc. mold and yeast phenotypes) or plant)) in which the transgene would otherwise be (partly) silenced. The viral RNA silencing suppressor/expressional enhancer of the present invention can also be used for enhancing the expression of homologous proteins, which can comprise the production of (therapeutic) monoclonal antibodies in hybridoma cells and for the production of metabolites synthesized by proteins, such as antibiotics and the like, if the expression of the homologous proteins would otherwise be (partly) silenced.

In another embodiment, the invention provides the use of a protein or fragment thereof or RNA molecule of a vertebrate virus which is capable of interfering with RNA silencing in an eukaryotic cell, i.e., an RNA silencing suppressor, to prevent or reverse silencing of the expression of transgenes in an eukaryotic cell, thereby ensuring that transgenes will be and remain expressed at high levels. Hence, the protein or RNA of the invention can be used to stabilize transgene expression.

In yet another embodiment, the invention provides the use of a protein or fragment thereof or RNA molecule of a vertebrate virus, which is capable of interfering with RNA silencing in a eukaryotic cell to enhance the expression of a heterologous nucleotide sequence in a eukaryotic cell. One can exploit the newfound knowledge about the mechanisms of RNA silencing suppression by vertebrate viruses as disclosed in the present invention to deliberately enhance/boost the expression of specific heterologous nucleotide sequences in a eukaryotic cell. For example, a nucleotide sequence of a vertebrate virus encoding a protein or RNA capable of interfering with RNA silencing in a eukaryotic cell can be used to prepare a nucleic acid construct for the transformation or transfection of a eukaryotic cell; which can be introduced into the eukaryotic cell simultaneously or separately from a nucleic acid construct comprising a heterologous nucleotide sequence of interest to be expressed in the eukaryotic cell, thus, ensuring enhancement of the expression of the heterologous nucleotide sequence of interest.

A eukaryotic cell can be derived from any eukaryotic organism, preferably an animal (including human), or plant or fungus (including both mold and yeast phenotypes). In a preferred embodiment, the invention provides the use of a protein or fragment thereof or RNA molecule of a plant or animal virus which is capable of interfering with RNA silencing in a fungal cell or in a cell from a mammalian cell-line to enhance/boost and/or stabilize the expression of a nucleotide sequence encoding a (pharmaceutical) protein or (industrial) enzyme, and the like.

In the examples, it is shown that the non-structural protein (NSs) of the genus Tospovirus within the Bunyaviridae, the non-structural protein (NS3) of the genus Tenuivirus and the non-structural protein (NS1) of the Orthomyxoviridae, the non-structural protein VP35 of the Filoviridae, and the non-structural protein E3L of the Poxviridae are capable of acting as RNA silencing suppressor proteins and also, thereby, as expressional enhancer proteins. Thus, at least all of these proteins are included in the definition of a protein from a vertebrate virus which is able to suppress silencing in a eukaryotic cell.

Also disclosed in the invention is that the NS 1 gene located on the viral complementary RNA strand of the smallest RNA segment of the Orthomyxoviridae and the NS_{S} gene and the NS3 gene are both located on the viral RNA strand of the ambisense third-largest RNA segment of the Tospoviruses and Tenuiviruses. Also disclosed in the invention is the NSP2 cistron which is located on the viral RNA strand of the Togaviridae.

Vertebrate viruses, according to the invention, comprise negative-strand RNA viruses, which can be viruses from the family Arenaviridae, Bunyaviridae, Orthomyxoviridae, Bornaviridae, Filoviridae, Paramyxoviridae, Rhabdoviridae, or the genus Tenuivirus; positive strand RNA viruses, which can be viruses from the family Picornaviridae, Flaviviridae, Togaviridae, Coronaviridae, Arteriviridae, Caliciviridae, Astroviridae or Retroviridae; double-strand RNA viruses, which can be viruses from the family Reoviridae; and DNA viruses, which can be viruses from the family Hepadnaviridae, Papovaviridae, Adenoviridae, Poxviridae or Herpesviridae.

In a preferred embodiment of the invention, the protein or fragment thereof of a vertebrate virus which is capable of interfering with RNA silencing in a eukaryotic cell comprises a viral non-structural protein. Since all negative-strand RNA viruses have a similar organization of the genome (being derived from one common ancestor) and all have at least one non-structural protein, it is believed that one of the functions of such a non-structural protein in these viruses is to increase the virulence of the cognate virus by suppressing a silencing mechanism in the host. Preferably, in negative-strand RNA viruses, the protein or fragment thereof is encoded by a gene present on the third largest RNA segment of the negative-strand RNA viral genome. For example, such a non-structural protein can be the non-structural protein (NSs) of the genus Tospovirus within the Bunyaviridae, the non-structural protein (NS3) of the genus Tenuivirus. Alternatively, the protein or fragment thereof is encoded by a gene present on the smallest RNA segment of the vertebrate viral genome, such as the non-structural (NS1) protein of the Orthomyxoviridae (preferably of Influenza virus A). Alternatively, the protein is a non-structural protein of the Filoviridae, preferably VP35 of Ebola virus. Thus, it is now possible to identify homologous proteins from other negative-strand RNA viruses (due to the close resemblance of their genomes), which can be tested for suppressor/enhancer activity in an assay of the invention.

Furthermore, a person skilled in the art is able to find similar proteins or RNA molecules from other viruses (or from other sources) in an assay of this invention, such as, for example, the non-structural protein of the Poxviridae, preferably E3L of Vaccinia virus.

In another preferred embodiment, the RNA silencing suppressor protein is an antibody or part thereof, directed against DICER or a RISC protein, thereby inactivating or inhibiting DICER or RISC.

In yet another preferred embodiment, the RNA silencing suppressor is an RNA molecule, including a virus-derived RNA molecule or a synthetic RNA molecule (RNA aptamer) having a secondary structure, usually containing foldings with one or more stretches of dsRNA, which is capable of binding to DICER or a RISC protein, thereby inactivating or inhibiting DICER or RISC.

In yet another preferred embodiment, the RNA silencing suppressor is a protein of non-viral origin able to scavenge siRNA by dsRNA binding and, thus, preventing the action of DICER or RISC or, a protein of eukaryotic origin involved in chromosome dynamics during mitosis and meiosis able to inhibit DICER or RISC.

A nucleic acid encoding a protein or RNA of the present invention which is capable of interfering with RNA silencing in an animal host cell can be used to immortalize primary animal cells. As the host RNA silencing machinery is responsible for suppression of the telomerase expression in mortal cells, the RNA silencing suppressors of the present invention enhance telomerase expression and prevent the replicative senescence that characterizes the Hayflick limit.

A nucleic acid encoding a protein or RNA of the present invention which is capable of interfering with RNA silencing in an animal host cell can be used for the production of virus particles, or particles of mutant or recombinant viral strains or viral vectors in animal host cells.

An animal host cell can be derived from any animal (including human). It can be derived from an existing animal cell line such as S2, Sf9, HeLa, CHO, HEK293, Vero, and the like, or it can be derived from a cell line of immortalized primary animal cells in which the immortal phenotype of the primary cells is conferred by expressing a protein or fragment thereof or RNA molecule of a virus (plant or animal) of the invention in the primary cells.

In another aspect, the invention provides a vaccine comprising a virus or a mutant or recombinant strain of a virus or a viral vector produced according to a method of the invention.

The invention currently provides a method to produce an attenuated virus in a host. The virus may be attenuated by preparing deletions in genes which may contribute to the virulence of the virus in a host. Methods to prepare deletions (e.g., point mutations, whole gene deletions, etc.) in a desired nucleic acid are known to those skilled in the art. The attenuated virus can be produced in host cells expressing a viral RNA silencing suppressor protein or RNA molecule, which enhances the production of viral particles in the host. The viral RNA silencing suppressor protein or RNA molecule can be derived from the same virus or from a heterologous virus. Since the produced virus is intact, it will be presented normally to the host immune system, which can react by building up resistance through its normal defense mechanism. Deletions of virulence genes can be combined with nucleic acid additions (i.e., antigenic/immunogenic sequences to boost mammalian cellular immune responses). Accordingly, the invention provides a method to produce a vaccine comprising an attenuated virus. The use of adjuvants in a vaccine of the invention to boost the mammalian cellular immune response is well known to those of skill in the art. Dosage and ways of administration of vaccines can be sorted out through normal clinical testing in so far as they are not yet available through the already registered vaccines.

In yet another embodiment, the invention provides the use of a protein or fragment thereof or RNA molecule of a virus which is capable of interfering with RNA silencing in an animal cell to suppress the silencing response of a host and/or to reverse the silencing of the expression of a nucleotide sequence in an animal cell once established. Thus, a RNA silencing suppressor of the present invention can be used for the improvement of packaging/producer cell lines for the production of recombinant virus particles or viral vectors, including retro-, lenti-, baculo-, adeno-, adeno-associated (AAV) or hybrid virus particles such as adeno-AAV viruses. This invention deals, preferably, with the production of recombinant virus particles, which harbor a nucleotide construct which is able to produce a single transcript or multiple transcripts capable of folding into double-stranded RNA. In a preferred embodiment of the invention, the protein or RNA molecule of the invention can be used for the improvement of packaging cell lines for the production of RNA-vectors, such as retro- or lentiviral vectors, harboring a nucleotide construct, which is able to produce a single transcript or multiple transcripts capable of folding into double-stranded RNA.

The invention further provides a nucleic acid construct comprising a nucleotide sequence encoding a protein or fragment thereof or RNA molecule of a virus which is capable of interfering with RNA silencing under the control of a suitable promoter for expression in a eukaryotic cell. For transcription from an expression construct (for transgenic in vivo transcription), a regulatory region such as a promoter, enhancer, splice donor and acceptor, or polyadenylation site may be used to transcribe the DNA. Suitable promoters are constitutive promoters (including virus-derived promoters), tissue-specific promoters, developmental promoters, inducible promoters, eukaryotic promoters and the like, including promoters that can be generated by one skilled in the art. The promoters can be of the DNA-dependent RNA-polymerase II (polII) type or of the DNA-dependent RNA-polymerase III (polIII) type. In case the RNA silencing suppressors of the invention are RNA molecules derived from DNA viruses, the nucleotide sequence(s) encoding the RNA molecules are preferably expressed in a eukaryotic cell under the control of an heterologous nucleotide sequence comprising the promoter, such that the expression of the RNA molecules are independent of the presence of other genes of the DNA viruses. Dependent on the gene delivery system and host utilized, any number of suitable transcription and translation elements, including constitutive and inducible promoters, may be used. The use and production of an expression construct are known in the art (vide WO 97/32016; U.S. Pat. No. 5,593,874; U.S. Pat. No. 5,698,425, U.S. Pat. No. 5,712,135; U.S. Pat. Nos. 5,789,214 and 5,804, 693). Methods for introducing nucleic acids into cells (e.g., by naked DNA transfer or by the use of gene delivery vehicles or vectors (of viral or non-viral origin etc.)), are known to a person skilled in the art. The simplest approach is naked DNA injection into local tissues or systemic circulation. Physical (gene gun, electroporation) and chemical (cationic lipid or polymer) approaches, and the like, have been utilized to improve the efficiency and target cell specificity of gene transfer by plasmid DNA. It is further possible to insert a heterologous nucleotide sequence of the present invention on a specific place in the genome of the target cell. This is accomplished by engineering the transgenic nucleotide sequence in between parts which are homologous to, preferably, non coding sequences. This nucleotide sequence will then be inserted on a specific place in the genome through homologous recombination. Using this mechanism, it is possible to insert the nucleotide sequence at a spot which is highly transcribed and where it does not disturb normal cell functions. The larger the length and the homology of the flanking sequences, the higher the efficiency of site-directed homologous recombination.

Another method to insert a heterologous nucleotide sequence of the present invention on a specific place in the genome of the target cell makes use of the capability of the AAV-1 p5IEE element (with or without the AAV-1 inverted terminal repeat, ITR) in combination with the AAV-1 rep 78/68 protein to integrate DNA site specifically into the AAVS1 site located on chromosome 19 of the human genome. (Philpott et al., Proc. Natl. Acad. Sci. U.S.A., 99: 12381-12385, 2002).

In a preferred embodiment, the protein or RNA of the invention is expressed in a eukaryotic cell from an episomal vector derived from a RNA or DNA virus. Suitable RNA viruses to be used as episomal vector include, but are not limited to, the Togaviridae, especially to the genus Alphavirus (such as Sindbis virus, Semliki Forest virus and Ross River virus). Suitable DNA viruses to be used as episomal vector include, but are not limited to, the Papovaviridae (such as BK virus, Simian virus 40 and Bovine papilloma virus-1) or Herpesviridae (such as the Epstein Barr virus). In another preferred embodiment, the protein or RNA of the invention is expressed in a eukaryotic cell from a mini-circle DNA vector, persistently replicating in the nucleus of the eukaryotic cell.

The use of a protein or RNA of the invention for the enhancement of expression in a variety of expression systems (e.g., eukaryotic systems (e.g., fungi, yeasts); baculovirus-insect cell systems; mammalian and plant systems and cell cultures, etc.)), known to one of skill in the art are encompassed by the present invention. One way this may be carried out is to express a natural, modified or recombinant nucleic acid of interest along with a nucleic acid encoding a protein of the invention in a single or separate construct under the control of the same or separate promoter, in a selected expression system. It is understood that the nucleic acid of interest may be ligated to the nucleic acid encoding a protein or fragment thereof of the invention to form a fusion protein. The two sequences may be cleaved by inclusion of a ‘cleavage’ sequence (e.g., sequence (2A) of the foot and mouth disease virus, and similar sequences) which encodes a posttranslational cleavage site between the two nucleic acid sequences. This sequence can mediate cleavage in a heterologous protein context in a range of eukaryotic expression systems. Alternatively, a RNA molecule of the invention can be cleaved from the mRNA comprising the nucleic acid of interest by ribozymes. Other novel or yet undisclosed means by which a nucleotide sequence encoding a protein or RNA molecule of a virus which is capable of interfering with RNA silencing in a host may be delivered alongside a nucleotide sequence(s) of interest to a host to the same location so that both sequences are under the same host genetic regulatory mechanisms are encompassed by the present invention.

In animals (including humans) a preferred method to introduce a nucleotide construct of the invention into a target cell is through the use of viral-based vectors. Preferred are viral vectors developed to prevent expression of immunogenic genes while encompassing all the genes responsible for proficient integration of the viral genome into that of the host. The nucleotide construct is engineered into a viral vector DNA molecule, which when packaged into a recombinant virus particle accomplishes efficient introduction of the nucleotide construct into the target cell. Most preferred is the use of viral vectors derived from adeno-associated (AAV), retroviruses, lentiviruses or adenovirus-AAV hybrid vectors. The methods for constructing such viral vectors and packing into a viral particle are well known to a person skilled in the art.

The invention further provides a gene delivery vehicle comprising a nucleic acid construct according to the invention. A gene delivery vehicle, as used herein, is any vehicle that can deliver nucleic acid to an organism. A gene delivery vehicle, or vector, can be of viral or non-viral origin. Viral vectors (RNA and DNA vectors), such as adeno-associated (AAV), retroviruses, lentiviruses, adenovirus-AAV hybrid vectors, and the like, are known to one of skill in the art. Non-viral vector systems such as liposomes, naked expression vector DNA, liposome-polycation complexes and peptide delivery systems, and the like, are encompassed by the invention. The invention further includes the use of yet undescribed biological and non biological based expression systems and novel host(s) systems that can be utilized to contain and express a nucleotide sequence encoding a protein of the invention (i.e., a protein of a virus which is capable of interfering with RNA silencing in a eukaryotic cell).

The invention further provides a eukaryotic cell harboring a nucleic acid construct encoding a protein or RNA molecule of the invention. Preferably, the eukaryotic cell expresses NSs of the genus Tospovirus within the Bunyaviridae, NS3 of the genus Tenuivirus, NS1 of the Orthomyxoviridae, VP35 of the Filoviridae and/or E3L of the Poxviridae.

As well, the invention provides a eukaryotic cell transformed or transfected with a gene delivery vehicle according to the invention. A suitable eukaryotic cell can be derived from any eukaryote which employs a mechanism of RNA silencing to “turn down” or “switch off” the expression of heterologous nucleotide sequences once introduced into the eukaryotic cell. Without being bound to theory, it is postulated that plants, fungi and animals “sense” the levels of specific RNA species (aberrant nucleic acid), and will activate the RNA silencing response (including PTGS and TGS) when these levels exceed a certain threshold. In plants, fungi and animals, the RNA silencing response is mediated by short, trans-acting molecules, siRNAs (short interfering RNAs). It is further postulated that RNA silencing is a mechanism which is widely used by eukaryotes, which means that any eukaryote could be considered to be a suitable host, according to the invention.

The invention is further explained with the aid of the following illustrative Examples.

### EXAMPLES

### Example 1

### Methods and Materials

Plant material & viruses:—Transgenic Nicotiana benthamiana plants were used harboring a GFP transgene expressed from a 35S promoter—NOS terminator expression cassette. Transgenic lines were selected for strong GFP fluorescence prior to self-pollination. Subsequent S1 plants were scored for gene silencing by checking for GFP expressing in meristematic tissues in otherwise non-expressing (silenced) plants. S2 progenies of these plants were homozygous and all showed a silenced phenotype resulting in silenced leaf tissue after several days and complete silencing also in veins and stems after several weeks. These S2 plants were used in a series of inoculation experiments. Tomato spotted wilt virus (TSWV) isolate BR-01, Groundnut ringspot virus (GRSV) isolate SA-05 and Impatiens necrotic spot virus (INSV) isolate NL-07 were inoculated in series on both GFP silenced and non-transgenic N. benthamiana plants acting as controls. For reference, the Potyviruses Potato virus Y (PVY) and Cowpea aphid-borne mosaic virus (CABMV) (Mlotschwa et al., Virus Genes 15:45-47, 2002) as well as two different Cucumber mosaic virus isolates (CMV-Lily and CMV-Alstroemeria, belonging to subgroup I and II, respectively, Chen et al., Arch. Virol. 146:1631-1636, 2001) were used in these experiments.

Inoculation was performed in the greenhouse in a 4-6 leaf developmental stage. Systemically infected top leaves were homogenized in 10 mM Naₓ(PO₄)_{y} pH 7.2 with 0.1% NaSO₃ added using a mortar and pestle. Each plant was inoculated on two leaves using carborundum powder as an abrasive agent. A sponge was used to apply the inocula on the leaf.

Agrobacterium clones and agro-infiltration:—An expression vector is used harboring an expression cassette consisting of the Cauliflower mosaic virus 35S promoter, the Tobacco mosaic virus 5′ untranslated region, a multiple cloning site and the nopaline synthase (nos) transcriptional terminator. The nucleoprotein N, movement protein NS_{M}, glycoprotein G1G2 precursor and NSs genes from Tomato spotted wilt virus have been cloned into this expression vector as described previously (Prins et al., Molecular Plant-Microbe Interactions 9:416-418, 1996). The coding sequences of the green fluorescent protein (GFP), of the HC-Pro protein from Cowpea aphid-born mosaic virus (CABMV) (Mlotshwa et al., Virus Genes 25: 207-16, 2002), of the 2b protein from Cucumber mosaic virus (CMV) subgroup I (Lucy et al., EMBO Journal 19: 1672-1680, 2000), of the NS3 gene from Rice Hoja Blanca virus (RHBV) (de Miranda et al., J. Gen. Virol. 75: 2127-32, 1994) and of the NS1 protein from Influenza virus A/PR/8/34 (IVA) are also cloned in this expression vector. A mutant form of NS1 denoted NS1rb is generated by PCR using primers EB08: 5′-d(GCGCTTCGCGCAGATCAGAAATCCC)-3′ (SEQ ID NO:______) and EB11: 5′-d(ATCAAGGAATGGGGCATCACCTAG)-3′ (SEQ ID NO:______). NS1rb has the R35A and K41A mutations presumably involved in dsRNA binding of the protein (Wang et al., RNA 5: 195-205, 1999). The NS1rb coding sequence is also cloned in this expression vector.

The expression cassettes carrying the individual genes are cloned in binary vector pBIN19 and subsequently introduced in Agrobacterium tumefaciens strain LBA4404 using tri-parental mating. The resulting DNA constructs are denoted pBIN-TSWV-N, pBIN-TSWV-NSm, pBIN-TSWV-G1G2, pBIN-TSWV-NSs, pBIN-GFP, pBIN-CABMV-HCPro, pBIN-CMV-2b, pBIN-RHBV-NS3, pBIN-IVA-NS1 and pBIN-IVA-NS1rb, respectively.

Agrobacterium T-DNA transient expression assays (ATTA) in N. benthamiana plants are performed by (co)-infiltrating at least two locations on the basal side of the leaf, with Agrobacterium suspensions using a 5 milliliters syringe without needle. Cultures are grown overnight at 28° C. from individual colonies in 2 milliliters YEB-medium (0.5% beef extract, 0.1% yeast extract, 0.5% peptone, 0.5% saccharose, 2 mM MgSO₄) incl. 20 micrograms per milliliter rifampicin and 50 micrograms per milliliter kanamycin. 400 microliters of cell culture is pelleted by centrifugation and resuspended in 2 milliliters of induction medium (10.5 g/l K₂HPO₄, 4.5 g/l KH₂PO₄, 1.0 g/l (NH₄)₂SO₄, 1 mM MgSO₄, 0.2% (v/v) glycerol, 50 μM acetosyringone and 10 mM MES pH5.6). After incubating overnight at 28° C. cells are pelleted again and washed in Murashige and Skoog (1962)-medium including 10 mM MES pH5.6. Cells are resuspended to a final OD₆₀₀ of 0.5 in MS-MES including 150 μM acetosyringone. Young, fully-expanded leaves are used for agro-infiltration and covered with plastic for 2-3 days in the greenhouse. Plants are subsequently monitored for GFP fluorescence using a handheld 125 W UV lamp (Philips HPW 125 W-T). Generally, expression reaches a stable level after 3-4 days.

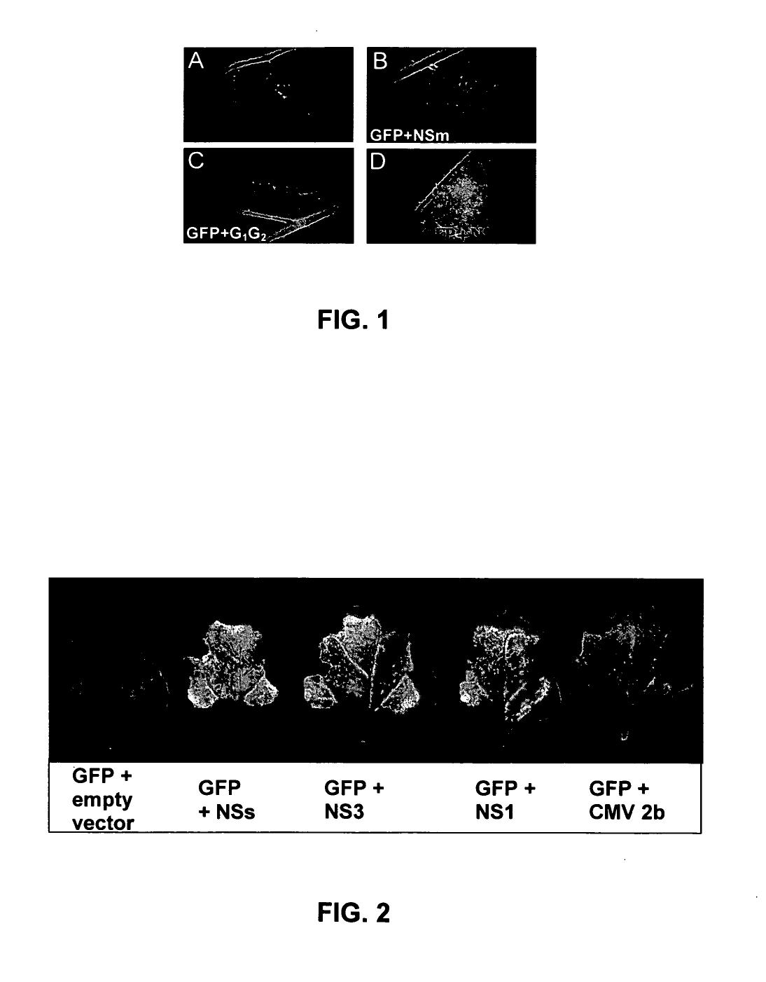

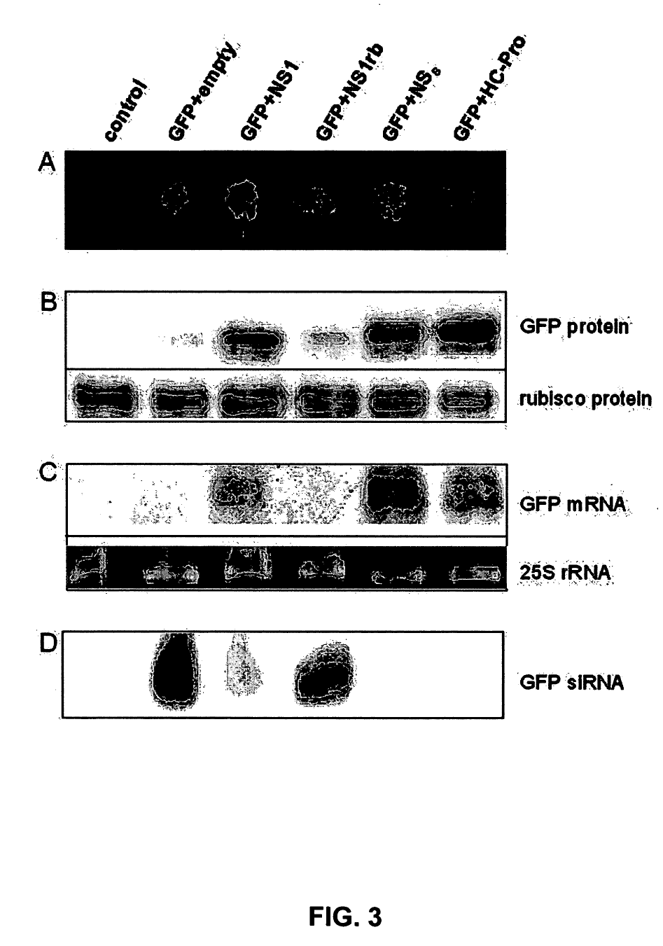

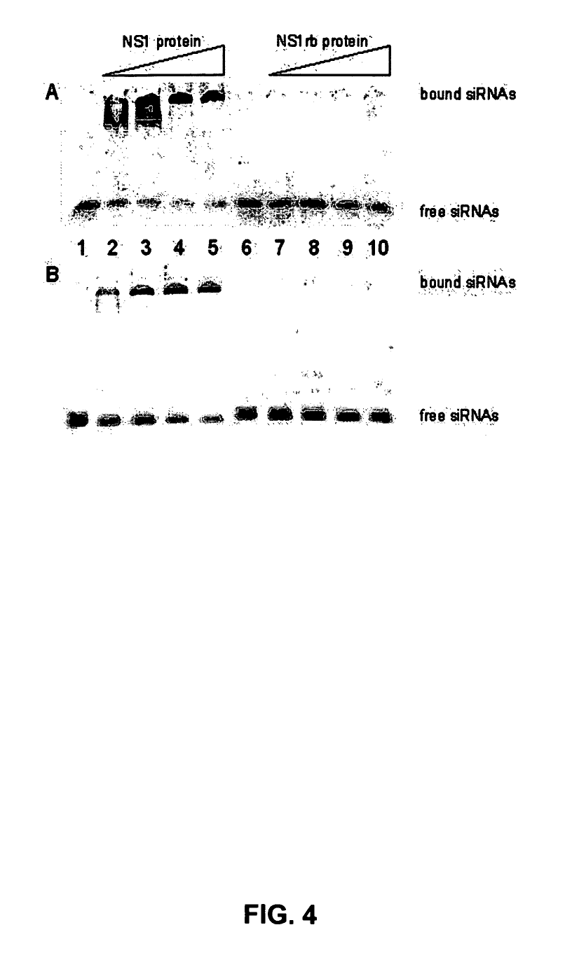

UV photography:—Pictures of whole plants (as shown in FIG. 1A) were made with a digital camera (Kodak DCS professional series) using a handheld 125 W ultraviolet lamp (Philips HPW 125 W-T) and 30 s exposure time. UV pictures at leaf level were made with 35 mm Kodak 200 ASA film using a black box carrying 2 small UV lamps (366 nm). Plants and leaves shown in FIGS. 1B and 1C: exposure time 2 min, using a Kodak Wratten no. 58 filter. Close-up UV pictures as shown in FIG. 2 were made with a digital camera (CoolSnap, combined red and green channel) using a binocular stereomicroscope (M3Z, Leica). The GFP imaging photographs of FIGS. 3 and 4A were taken with a yellow 022 B+W filter from Proline. Variable exposure times were used, depending on the intensity of the fluorescence.

Molecular analyses:—Northern blot analyses were performed using standard protocols using ³²P radiolabeled PCR products. Western blot analysis of GFP and NS_{S} was performed using polyclonal rabbit antiserum.

Isolation and enrichment of small RNAs was performed as described by Hamilton and Baulcombe, Science 286:950-952, 1999. Detection of siRNAs was performed by RnaseA/T1 protection assays according to Sijen et al., Cell 107:1558-1560, 1997.

### Example 2

### Agrobacterium T-DNA Transient Expression Assays in N. benthamiana

The Agrobacterium strains carrying TSWV gene constructs are injected in leaves together with the strain carrying pBIN-GFP. The GFP expression is monitored during the following days and photographed 6 days after injection (FIG. 1). Only co-infiltration of pBIN-GFP with pBIN-TSWV-NS_{S} gene leads to an increase of the GFP fluorescence in the injected leaf areas (FIG. 1D). Co-infiltration of pBIN-GFP with the other TSWV gene constructs does not lead to an increase of the GFP fluorescence in the injected leaf areas (FIGS. 1A, B, C).

Co-infiltration of pBIN-GFP with pBIN-RHBV-NS3, pBIN-IVA-NS1 or pBIN-CABMV-HCPro also leads to an increase in the GFP, which is much stronger than that obtained with pBIN-CMV-2b (FIGS. 2 and 3A).

### Example 3

### Influenza Virus A NS1 Binds siRNAs and Protects GFP mRNAs from Degradation

Nicotiana benthamiana leaves are co-infiltrated with the Agrobacterium strain harboring pBIN-GFP and those harboring pBIN-IVA-NS1, pBIN-IVA-NS1rb, pBIN-TSWV-NSs or pBIN-CABMV-HCPro (FIG. 3A). The amount of GFP accumulating in the infiltrated leaf sectors is determined using quantitative Western blot analysis. Total protein is extracted from infiltrated leaf sectors and resolved using denaturating polyacrylamide gel electrophoresis. The proteins are blotted to nitrocellulose and the ribulose 1,5 bi-phosphate carboxylase (rubisco) protein abundance on the blots is visualized using anti-rubisco antibodies and used as a loading control. The amounts of expressed GFP is visualized using anti-GFP antibodies. The amounts of GFP accumulating in cells which also express TSWV NSs, CABMV HC-Pro or WVA NS1 are significantly higher than those in cells which also express the pBIN19 empty vector or pBIN-WVA-NS1rb (FIG. 3B). To verify that high GFP expression is indeed due to mRNA protection rather than to an enhanced translation, Northern blot analyses of mRNA purified from infiltrated leaf sectors are performed. The blots are probed with a DIG-labeled (Boehringer) GFP-specific DNA fragment. Ethidium bromide staining of the same gel shows the 25S ribosomal RNA as a loading control. The high accumulation of GFP mRNAs in leaf sectors co-infiltrated with Agrobacterium strains harboring pBIN-GFP and those harboring pBIN-TSWV-NSs, pBIN-CABMV-HCPro or pBIN-IVA-NS1 but not the pBIN19 empty vector or pBIN-IVA-NS1rb, is caused by protection of the GFP mRNA from degradation (FIG. 3C).

To detect GFP-specific siRNAs, leaf material from infiltrated sectors is ground in liquid nitrogen and dissolved in 1.3 milliliters of 2% Sarkosyl, 5M NaCl per gram of leaf material. After phenol extraction, polysaccharide contaminants are precipitated with 1 volume 3M ammonium acetate (pH 5.2). The water phase is ethanol precipitated and dissolved in TE (10 mM Tris-HCl, 1 mM EDTA pH=7.5). To remove larger RNA molecules, a poly-ethylene glycol (PEG) precipitation is performed using 5% PEG8000/0.5M NaCl final concentration. The supernatant, containing the siRNAs is precipitated with ethanol. Twenty micrograms of total siRNAs per sample and 10 micrograms total RNA of the PEG8000 precipitate are analyzed by Northern blot using a GFP specific DIG labeled DNA fragment to detect the GFP siRNAs and mRNAs, respectively. GFP-specific siRNAs are present in the leaf sectors expressing GFP and the pBIN19 empty vector or IVA-NS1rb. The GFP-specific siRNAs are absent in the leaf sectors expressing GFP and the IVA NS1, TSWV NSs or CABMV HC-Pro.

To verify whether VA NS1 is capable of binding siRNAs, gel retardation studies of siRNAs binding to increasing amounts of NS1 protein were performed. The NS1 and NS1rb proteins were expressed in an N-terminally his-tagged form using the pQE31 vector system (Qiagen). The proteins were purified on TALON CellThru affinity columns (BD Biosciences). The GL3 siRNA molecules (Hohjoh, FEBS Lett. 521:195-199, 2002) with the sequence 5′-r(CUUACGCUGAGUACUUCGA)d(TT)-3′ (SEQ ID NO:______) and 5′-r(UCGAAGUACUCAGCGUAAG)d(TT)-3′ (SEQ ID NO:______) directed against the Photinus pyralis luciferase gene are purchased from Qiagen. The 5′-phosphate groups of the GL3 luciferase siRNAs are removed by phosphatase treatment and replaced by ³³P radiolabeled phosphate groups by polynucleotide kinase treatment. Plant siRNAs are enriched, radiolabeled with ³³P and purified from an 8% polyacrylamide gel using the radiolabeled luciferase siRNAs as a size marker. Radiolabeled synthetic GL3 siRNAs are incubated on ice for 20 minutes with increasing amounts of purified NS1 and NS1rb proteins. A non-denaturating 5% polyacrylamide gel is used for sample analysis (Wang et al., RNA 5: 195-205, 1999). The radiolabeled siRNAs are detected by autoradiography. In addition, the radiolabeled plant siRNAs are incubated and visualized under the same conditions. IVA NS1 is able to bind radiolabeled synthetic luciferase siRNAs resulting in band shifting. Similarly, NS1 is capable of binding radiolabeled siRNAs extracted from plants. The NS1 protein with mutations in the RNA binding domain, NS1rb, is not able to bind synthetic luciferase siRNAs or siRNAs extracted from plants (FIG. 3D).

The IVA NS1 protein is capable of enhancing GFP expression by protecting the GFP mRNAs from degradation by the RNA silencing machinery. The double-stranded RNA-binding capacity of the protein is crucial for the expressional enhancer or RNA silencing suppressor activity, since a mutant protein, NS1rb, which is not able to bind double-stranded RNA including siRNAs cannot protect the GFP mRNA from degradation and is not capable of enhancing GFP expression.

### Example 4

### Construction of Mammalian Expression Vectors and of siRNA Molecules

The coding sequences of the NS3 protein from RHBV, the NSs protein from Rift Valley fever virus (RVFV), of the VP35 protein from Ebola virus (EV), of the E3L protein from Vaccinia virus (VV), of the NS1 protein from IVA, of the IVA NS1 mutant NS1rb protein and of the Luciferase proteins from Photinus pyralis (Pluc) and Renilla reniformis (Rluc) (purchased from Promega) are cloned into pEF5/FRT/V5-DEST using ‘gateway’ (Invitrogen) following the manufacturer's recommendations. The expression vectors are denoted pRHBV-NS3, pRVFV-NSs, pEV-VP35, pVV-E3L, pIVA-NS1, pIVA-NS1rb, pPluc and pRluc, respectively.

### Example 5

### Production of HEK293/Flp-In Cell Lines Expressing Influenza Virus A NS1 and Photinus pyralis Luciferase

HEK293/Flp-In human embryonic kidney cells with a single FRT recombination site in their chromosomal DNA (Graham et al., 1977. J. Gen Virol. 36:59-74; purchased from Invitrogen) are grown at 37 degrees Celsius in Dulbecco's modified Eagle medium with 0.11 grams per liter sodium pyridoxine, MEM non-essential amino acids (Gibco), supplemented with 10% Fetal bovine serum (Biochrom KG) and 100 micrograms per milliliter Zeocine (Invitrogen), 100 units per milliliter penicillin and 100 micrograms per milliliter streptomycin (DME medium). Twice a week the confluent cell cultures are diluted 10 times in DME medium and sub-cultured at 37 degrees Celsius. Plasmid DNA of pIVA-NS1 and pPluc are recombined into the FRT recombination site of HEK293/Flp-In cells according to the manufacturer's recommendations (Invitrogen). Cell batches with pIVA-NS1 DNA and cell batches with pPluc DNA stably integrated into the FRT recombination site on the chromosomal DNA are grown and selected in appropriate medium provided with 100 micrograms per milliliter hygromycin and are denoted HEK293:NS1 and HEK293:Pluc, respectively.

HEK293:NS1 and HEK293:Pluc cell cultures are grown normally at 37° C. in DME medium and sub-cultured twice a week, just as the HEK293/Flp-In cell cultures.

### Example 6

### Transfection of Mammalian Cells

Three milliliters of a confluent HEK293/Flp-In cell culture is co-transfected with Pluc and increasing amounts of pEF5/FRT/V5-DEST empty vector, pRHBV-NS3, pRVFV-NSs, pEV-VP35, pVV-E3L, pIVA-NS1 or pIVA-NS2rb DNA using the lipofectamin2000 method, following the manufacturer's recommendations (Invitrogen). The luciferase activities are quantified using the dual-luciferase reporter assay from Promega. RHBV NS3, EV VP35, VV E3L and IVA NS1 are capable of enhancing the Pluc expression in a concentration dependent manner. The pEF5/FRT/V5-DEST empty vector and RVFV NSs are not able to enhance the Pluc expression, whereas IVA NS1rb enhances the Pluc expression to a lesser extent than the wild type NS1 protein.

Three milliliters of a confluent HEK293:Pluc cell culture is co-transfected with pRluc and GL3 siRNAs, together with increasing amounts of pEF5/FRT/V5-DEST, pRHBV-NS3, pRVFV-NSs, pEV-VP35, pVV-E3L, pIVA-NS1 or pVA-NS1rb DNA using the lipofectamin2000 method. Co-transfection of pRluc DNA, GL3 siRNAs and pEF5/FRT/V5-DEST DNA leads to a significant reduction in Pluc expression. Co-transfection of pRluc DNA, GL3 siRNAs and pRHBV-NS3, pEV-VP35, pVV-E3L or pIVA-NS1 DNA leads to a restoration of the Pluc expression. In addition, the Rluc expression is boosted in the presence of DNA of these expression vectors. RHBV NS3, EV VP35, VV E3L and IVA NS1 are RNA silencing suppressors or expressional enhancers. pRVFV-NSs is not able to restore Pluc expression or to boost Rluc expression and, therefore, has no RNA silencing suppressor activity. Similar to the plant assay, pIVA-NS1rb is significantly less active than pIVA-NS1 in animal cells.

### Example 7

### Production of HIV-1 and VSV Particles in HEK293 and HEK293:NS1 Cells

Three milliliters of confluent HEK293/Flp-In and HEK293:NS1 cell cultures with an integrated pIVA-NS1 expression vector are transfected with 1 μg of pLai DNA (Peden et al., 1991. Virology 185:661-672) using lipofectamine according to the manufacturer's recommendation's (Invitrogen) and incubated at 37° C. Seven days post infection, the amount of virus in the supernatants is quantified using p24 ELISA and the “tissue culture infective dose 50%” (TCID₅₀) values are determined by titration of dilutions of the supernatants to confluent SupT1 human non-Hodgkin's T-lymphoma cell cultures (Smith et al., Cancer Research 44:5657, 1984). The amount of virus in the supernatant of the HEK293:NS1 cells harboring the NS1 plasmid is significantly higher than that in the supernatant of the HEK293/Flp-In cells.

Three milliliters of confluent HEK293/Flp-In and HEK293:NS1 cell cultures are infected with Vesicular Stomatitis virus (VSV) strain San Juan A, at a “moiety of infection” (MOI) of 5. After three days, incubation at 37° C., the TCID₅₀ values are determined by titration of dilutions of the supernatants to confluent HEK293/Flp-In cell cultures. The HEK293:NS1 cells are significantly more permissive to VSV infection than the HEK293/Flp-In cells. Moreover, VSV grows at higher titers in HEK293:NS1 cells than in HEK293/Flp-In cells.

### Example 8

### Production of Recombinant Lentiviral Particles in HEK293FT Cells

The CMV promoter, the GATEWAY cassette and V5 epitope DNA fragment of plasmid pLenti6/V5-DEST (Invitrogen) were removed and replaced with a short linker DNA fragment with AscI and PacI restriction sites, yielding pLenti6/Asc/Pac. An expression vector denoted pWdV22, harboring a gene cassette comprising the EF1-alpha promoter, a 300 base pairs antisense Nef DNA fragment, a 350 base pairs sense Nef DNA fragment and the bovine growth hormone poly-adenylation signal (the dsNef gene cassette. See patent application EP02076434.6) was digested with HindIII and an AscI-HindIII adapter DNA fragment was ligated. The dsNef gene cassette was released from pWdV22 by digestion with PacI and cloned in AscI/PacI digested pLenti6/Asc/Pac, yielding pLenti/dsNef. Recombinant lentivirus particles harboring pLenti/dsNef were produced in HEK 293FT cells (Invitrogen) using the lentiviral packaging plasmids pLP1 (gag/pol), pLP2 (rev) and pLP/VSV-G (the VSV membrane glycoprotein gene) purchased from Invitrogen, according to the manufacturer's recommendations. Recombinant virus particles could not be produced in reasonable amounts in HEK293FT cells unless a plasmid with a RNA silencing suppressor from Example 6 was co-transfected into the HEK293FT cells, which indicates that the presence of a RNA silencing suppressor in the producer cell is crucial for the production of pLenti/dsNef recombinant virus particles.

1. A method of prevent silencing expression of a nucleotide sequence in a eukaryotic cell, the method comprising:
introducing into the eukaryotic cell a peptide or RNA molecule of a vertebrate virus that interferes with RNA silencing in the eukaryotic cell.

2. A method of reversing silencing of expression of a nucleotide sequence in a eukaryotic cell, once established, said method comprising:
introducing into the eukaryotic cell a peptide or RNA molecule of a vertebrate virus that interferes with RNA silencing in the eukaryotic cell.

3. A method of enhancing, boosting, and/or stabilizing the production or manufacture of a protein or a metabolite synthesized by proteins in a eukaryotic cell, said method comprising:
introducing into the eukaryotic cell a peptide or RNA molecule of a vertebrate virus that interferes with RNA silencing in the eukaryotic cell.

4. A method of producing or manufacturing a vertebrate virus, mutant thereof, or recombinant strain thereof or a viral vector in an animal cell, said method comprising:
introducing into the animal cell a peptide or RNA molecule of a vertebrate virus that interferes with RNA silencing in said animal cell.

5. The method according to claim 4, wherein said vertebrate virus, comprises a virus selected from the group of families Arenaviridae, Bunyaviridae, Orthomyxoviridae, Bornaviridae, Filoviridae, Paramyxoviridae, Rhabdoviridae, or the genus Tenuivirus, the family Picornaviridae, Flaviviridae, Togaviridae, Coronaviridae, Arteriviridae, Caliciviridae, Astroviridae, Arteriviridae, Herpesviridae, Reoviridae, Adenoviridae, Papovaviridae, and Poxviridae.

6. The method according to claim 5, wherein said peptide comprises a viral non-structural protein.

7. The method according to claim 6, wherein said viral non-structural protein comprises the non-structural protein (NSs) of genus Tospovirus within Bunyaviridae, non-structural protein (NS3) of Tenuiviruses, non-structural protein (NSI) of Orthomyxoviridae, a non-structural protein of influenza virus A, a non-structural protein (VP35) of Filoviridae, a non-structural protein of Ebola virus, a non-structural protein (E3L) of Poxviridae, or non-structural protein of Vaccinia virus.

8. A nucleic acid construct comprising:
a nucleotide sequence encoding a peptide or RNA molecule of a virus that interferes with RNA silencing in a eukaryotic cell, said nucleotide sequence under control of a heterologous promoter for expression in a eukaryotic cell.

9. A gene delivery vehicle comprising the nucleic acid construct of claim 8.

10. A eukaryotic cell transformed with the gene delivery vehicle of claim 9.

11. The method according to claim 4, wherein the peptide or RNA molecule is heterologous to the virus produced.

12. The method according claim 4 wherein the virus, mutant or recombinant strain thereof or a viral vector thus produced belongs to or is derived from the Arenaviridae, Bunyaviridae, Orthomyxoviridae, Paramyxoviridae, Filoviridae, Rhabdoviridae, Coronaviridae, Picornaviridae, Flaviviridae, Togaviridae, Retroviridae, Adenoviridae, Herpesviridae, Hepadnaviridae or Papovaviridae.

13. The method according to claim 4, wherein said virus lacks the functional nucleotide coding for the protein or fragment thereof or RNA which is capable of interfering with RNA silencing in eukaryotic cells.

14. The method according to claim 4 to produce recombinant virus particles, which harbor a nucleotide construct able to produce a single transcript or multiple transcripts capable of folding into double-stranded RNA.

15. A method to immortalize primary cells comprising:
introducing into said primary cell a peptide or RNA molecule of a virus able to interfere with RNA silencing, thereby enhancing the telomerase activity in said cell.

16. An immortalized primary cell or a cell line made of immortalized primary cells produced by the method of claim 15.

17. An assay for identifying a peptide or RNA molecule of a virus able to interfere with RNA silencing in a eukaryotic cell, said assay comprising:
introducing simultaneously or separately into a eukaryotic cell:
(a) a nucleic acid construct comprising a nucleotide sequence encoding a protein or RNA molecule of a virus; and
(b) a nucleic acid construct comprising a nucleotide sequence encoding a reporter protein into a eukaryotic cell silenced in the expression of a nucleic acid encoding said reporter protein, and
detecting the suppression/dampening/reversal of silencing and/or a boost in reporter protein expression.

18. The assay of claim 17, wherein the nucleic acids are introduced into a mammalian cell.

19. The method according to claim 1, wherein said vertebrate virus, comprises a virus selected from the group of families Arenaviridae, Bunyaviridae, Orthomyxoviridae, Bornaviridae, Filoviridae, Paramyxoviridae, Rhabdoviridae, or the genus Tenuivirus, the family Picornaviridae, Flaviviridae, Togaviridae, Coronaviridae, Arteriviridae, Caliciviridae, Astroviridae, Arteriviridae, Herpesviridae, Reoviridae, Adenoviridae, Papovaviridae, and Poxviridae.

20. The method according to claim 2, wherein said vertebrate virus, comprises a virus selected from the group of families Arenaviridae, Bunyaviridae, Orthomyxoviridae, Bornaviridae, Filoviridae, Paramyxoviridae, Rhabdoviridae, or the genus Tenuivirus, the family Picornaviridae, Flaviviridae, Togaviridae, Coronaviridae, Arteriviridae, Caliciviridae, Astroviridae, Arteriviridae, Herpesviridae, Reoviridae, Adenoviridae, Papovaviridae, and Poxviridae.
